SEQUENCE LISTING (15 sequences)

SEQ ID NO: 1 - DNA, 102 bp, Streptococcus pyogenes
gcggatttcgtggatgggcagttttagagctagaaatagcaagttaaaataaggctagtccgttatcaacttgaaaaagtggcaccgagtcggtgctttttt

SEQ ID NO: 2 - RNA, 103 nt, streptococcus pyogenes
gtcttggtgctgatgtcggcagttttagagctagaaatagcaagttaaaataaggctagtccgttatcaacttgaaaaagtggcaccgagtcggtgctttttt

SEQ ID NO: 3 - RNA, 102 nt, Streptococcus pyogenes
gaccatgattacggattcacgttttagagctagaaatagcaagttaaaataaggctagtccgttatcaacttgaaaaagtggcaccgagtcggtgctttttt

SEQ ID NO: 4 - RNA, 105 nt, streptococcus pyogenes
ggccagtgaatccgtaatcagttttagagctagaaatagcaagttaaaataaggctagtccgttatcaacttgaaaaagtggcaccgagtcggtgcttttttttt

SEQ ID NO: 5 - protein, 4107 aa, Streptococcus pyogenes
ATGGATAAGAAATACTCAATAGGCTTAGATATCGGCACAAATAGCGTCGGATGGGCGGTGATCACTGATGAATATAAGGTTCCGTCTAAAAAGTTCAAGGTTCTGGGAAATACAGACCGCCACAGTATCAAAAAAAATCTTATAGGGGCTCTTTTATTTGACAGTGGAGAGACAGCGGAAGCGACTCGTCTCAAACGGACAGCTCGTAGAAGGTATACACGTCGGAAGAATCGTATTTGTTATCTACAGGAGATTTTTTCAAATGAGATGGCGAAAGTAGATGATAGTTTCTTTCATCGACTTGAAGAGTCTTTTTTGGTGGAAGAAGACAAGAAGCATGAACGTCATCCTATTTTTGGAAATATAGTAGATGAAGTTGCTTATCATGAGAAATATCCAACTATCTATCATCTGCGAAAAAAATTGGTAGATTCTACTGATAAAGCGGATTTGCGCTTAATCTATTTGGCCTTAGCGCATATGATTAAGTTTCGTGGTCATTTTTTGATTGAGGGAGATTTAAATCCTGATAATAGTGATGTGGACAAACTATTTATCCAGTTGGTACAAACCTACAATCAATTATTTGAAGAAAACCCTATTAACGCAAGTGGAGTAGATGCTAAAGCGATTCTTTCTGCACGATTGAGTAAATCAAGACGATTAGAAAATCTCATTGCTCAGCTCCCCGGTGAGAAGAAAAATGGCTTATTTGGGAATCTCATTGCTTTGTCATTGGGTTTGACCCCTAATTTTAAATCAAATTTTGATTTGGCAGAAGATGCTAAATTACAGCTTTCAAAAGATACTTACGATGATGATTTAGATAATTTATTGGCGCAAATTGGAGATCAATATGCTGATTTGTTTTTGGCAGCTAAGAATTTATCAGATGCTATTTTACTTTCAGATATCCTAAGAGTAAATACTGAAATAACTAAGGCTCCCCTATCAGCTTCAATGATTAAACGCTACGATGAACATCATCAAGACTTGACTCTTTTAAAAGCTTTAGTTCGACAACAACTTCCAGAAAAGTATAAAGAAATCTTTTTTGATCAATCAAAAAACGGATATGCAGGTTATATTGATGGGGGAGCTAGCCAAGAAGAATTTTATAAATTTATCAAACCAATTTTAGAAAAAATGGATGGTACTGAGGAATTATTGGTGAAACTAAATCGTGAAGATTTGCTGCGCAAGCAACGGACCTTTGACAACGGCTCTATTCCCCATCAAATTCACTTGGGTGAGCTGCATGCTATTTTGAGAAGACAAGAAGACTTTTATCCATTTTTAAAAGACAATCGTGAGAAGATTGAAAAAATCTTGACTTTTCGAATTCCTTATTATGTTGGTCCATTGGCGCGTGGCAATAGTCGTTTTGCATGGATGACTCGGAAGTCTGAAGAAACAATTACCCCATGGAATTTTGAAGAAGTTGTCGATAAAGGTGCTTCAGCTCAATCATTTATTGAACGCATGACAAACTTTGATAAAAATCTTCCAAATGAAAAAGTACTACCAAAACATAGTTTGCTTTATGAGTATTTTACGGTTTATAACGAATTGACAAAGGTCAAATATGTTACTGAAGGAATGCGAAAACCAGCATTTCTTTCAGGTGAACAGAAGAAAGCCATTGTTGATTTACTCTTCAAAACAAATCGAAAAGTAACCGTTAAGCAATTAAAAGAAGATTATTTCAAAAAAATAGAATGTTTTGATAGTGTTGAAATTTCAGGAGTTGAAGATAGATTTAATGCTTCATTAGGTACCTACCATGATTTGCTAAAAATTATTAAAGATAAAGATTTTTTGGATAATGAAGAAAATGAAGATATCTTAGAGGATATTGTTTTAACATTGACCTTATTTGAAGATAGGGAGATGATTGAGGAAAGACTTAAAACATATGCTCACCTCTTTGATGATAAGGTGATGAAACAGCTTAAACGTCGCCGTTATACTGGTTGGGGACGTTTGTCTCGAAAATTGATTAATGGTATTAGGGATAAGCAATCTGGCAAAACAATATTAGATTTTTTGAAATCAGATGGTTTTGCCAATCGCAATTTTATGCAGCTGATCCATGATGATAGTTTGACATTTAAAGAAGACATTCAAAAAGCACAAGTGTCTGGACAAGGCGATAGTTTACATGAACATATTGCAAATTTAGCTGGTAGCCCTGCTATTAAAAAAGGTATTTTACAGACTGTAAAAGTTGTTGATGAATTGGTCAAAGTAATGGGGCGGCATAAGCCAGAAAATATCGTTATTGAAATGGCACGTGAAAATCAGACAACTCAAAAGGGCCAGAAAAATTCGCGAGAGCGTATGAAACGAATCGAAGAAGGTATCAAAGAATTAGGAAGTCAGATTCTTAAAGAGCATCCTGTTGAAAATACTCAATTGCAAAATGAAAAGCTCTATCTCTATTATCTCCAAAATGGAAGAGACATGTATGTGGACCAAGAATTAGATATTAATCGTTTAAGTGATTATGATGTCGATCACATTGTTCCACAAAGTTTCCTTAAAGACGATTCAATAGACAATAAGGTCTTAACGCGTTCTGATAAAAATCGTGGTAAATCGGATAACGTTCCAAGTGAAGAAGTAGTCAAAAAGATGAAAAACTATTGGAGACAACTTCTAAACGCCAAGTTAATCACTCAACGTAAGTTTGATAATTTAACGAAAGCTGAACGTGGAGGTTTGAGTGAACTTGATAAAGCTGGTTTTATCAAACGCCAATTGGTTGAAACTCGCCAAATCACTAAGCATGTGGCACAAATTTTGGATAGTCGCATGAATACTAAATACGATGAAAATGATAAACTTATTCGAGAGGTTAAAGTGATTACCTTAAAATCTAAATTAGTTTCTGACTTCCGAAAAGATTTCCAATTCTATAAAGTACGTGAGATTAACAATTACCATCATGCCCATGATGCGTATCTAAATGCCGTCGTTGGAACTGCTTTGATTAAGAAATATCCAAAACTTGAATCGGAGTTTGTCTATGGTGATTATAAAGTTTATGATGTTCGTAAAATGATTGCTAAGTCTGAGCAAGAAATAGGCAAAGCAACCGCAAAATATTTCTTTTACTCTAATATCATGAACTTCTTCAAAACAGAAATTACACTTGCAAATGGAGAGATTCGCAAACGCCCTCTAATCGAAACTAATGGGGAAACTGGAGAAATTGTCTGGGATAAAGGGCGAGATTTTGCCACAGTGCGCAAAGTATTGTCCATGCCCCAAGTCAATATTGTCAAGAAAACAGAAGTACAGACAGGCGGATTCTCCAAGGAGTCAATTTTACCAAAAAGAAATTCGGACAAGCTTATTGCTCGTAAAAAAGACTGGGATCCAAAAAAATATGGTGGTTTTGATAGTCCAACGGTAGCTTATTCAGTCCTAGTGGTTGCTAAGGTGGAAAAAGGGAAATCGAAGAAGTTAAAATCCGTTAAAGAGTTACTAGGGATCACAATTATGGAAAGAAGTTCCTTTGAAAAAAATCCGATTGACTTTTTAGAAGCTAAAGGATATAAGGAAGTTAAAAAAGACTTAATCATTAAACTACCTAAATATAGTCTTTTTGAGTTAGAAAACGGTCGTAAACGGATGCTGGCTAGTGCCGGAGAATTACAAAAAGGAAATGAGCTGGCTCTGCCAAGCAAATATGTGAATTTTTTATATTTAGCTAGTCATTATGAAAAGTTGAAGGGTAGTCCAGAAGATAACGAACAAAAACAATTGTTTGTGGAGCAGCATAAGCATTATTTAGATGAGATTATTGAGCAAATCAGTGAATTTTCTAAGCGTGTTATTTTAGCAGATGCCAATTTAGATAAAGTTCTTAGTGCATATAACAAACATAGAGACAAACCAATACGTGAACAAGCAGAAAATATTATTCATTTATTTACGTTGACGAATCTTGGAGCTCCCGCTGCTTTTAAATATTTTGATACAACAATTGATCGTAAACGATATACGTCTACAAAAGAAGTTTTAGATGCCACTCTTATCCATCAATCCATCACTGGTCTTTATGAAACACGCATTGATTTGAGTCAGCTAGGAGGTGACTGA

SEQ ID NO: 6 - protein, 1368 aa, Streptococcus pyogenes [sequence omitted]

SEQ ID NO: 7 - protein, 2280 aa, Mycobacterium tuberculosis
ATGGGTTCGGCGTCGGAGCAACGGGTGACGCTGACCAACGCCGACAAGGTGCTCTATCCCGCCACCGGGACCACAAAGTCCGATATCTTCGACTACTACGCCGGTGTTGCCGAAGTCATGCTCGGCCACATCGCGGGACGGCCGGCGACGCGCAAGCGCTGGCCTAACGGCGTCGACCAACCCGCGTTCTTCGAAAAGCAGTTGGCGTTGTCGGCGCCGCCTTGGCTGTCACGTGCAACGGTGGCGCACCGGTCCGGGACGACGACCTATCCGATCATCGATAGCGCAACCGGGCTGGCCTGGATCGCCCAACAGGCGGCGCTGGAGGTGCACGTGCCGCAGTGGCGGTTTGTCGCCGAGCCCGGATCAGGTGAGTTAAATCCGGGCCCGGCAACGCGTTTGGTGTTCGACCTGGACCCGGGCGAAGGCGTGATGATGGCCCAGCTGGCCGAGGTGGCGCGCGCGGTTCGTGATCTTCTCGCCGATATCGGGTTGGTCACCTTCCCGGTCACCAGCGGCAGCAAGGGATTGCATCTGTACACACCGCTGGATGAGCCGGTGAGCAGCAGGGGAGCCACGGTGTTGGCCAAGCGCGTCGCGCAGCGATTGGAGCAGGCGATGCCCGCGTTGGTCACCTCGACCATGACCAAAAGCCTGCGGGCCGGGAAGGTGTTTGTGGACTGGAGCCAGAACAGCGGCTCGAAGACCACCATCGCGCCGTACTCACTACGTGGCCGGACGCATCCGACCGTCGCGGCGCCACGCACCTGGGCGGAGCTCGACGACCCCGCACTGCGTCAGCTCTCCTACGACGAGGTGCTGACCCGGATTGCCCGCGACGGCGATCTGCTCGAGCGGCTGGATGCCGACGCTCCGGTAGCGGACCGGTTGACCCGATACCGCCGCATGCGCGACGCATCGAAAACTCCCGAGCCGATTCCCACGGCGAAACCCGTTACCGGAGACGGCAATACGTTCGTCATCCAGGAGCATCACGCGCGTCGGCCGCACTACGATTTCCGGCTGGAATGCGACGGCGTGCTGGTCTCGTGGGCGGTACCGAAAAACCTGCCCGACAACACATCGGTTAACCATCTAGCGATACACACCGAGGACCACCCGCTGGAATACGCCACGTTCGAGGGCGCGATTCCCAGCGGGGAGTACGGCGCCGGCAAGGTGATCATCTGGGACTCCGGCACTTACGACACCGAGAAGTTCCACGATGACCCGCACACGGGGGAGGTCATCGTGAATCTGCACGGCGGCCGGATCTCTGGGCGTTATGCGCTGATTCGGACCAACGGCGATCGGTGGCTGGCGCACCGCCTAAAGAATCAGAAAGACCAGAAGGTGTTCGAGTTCGACAATCTGGCCCCAATGCTTGCCACGCACGGCACGGTGGCCGGTCTAAAGGCCAGCCAGTGGGCGTTCGAAGGCAAGTGGGACGGCTACCGGTTGCTGGTTGAGGCTGACCACGGCGCCGTGCGGCTGCGGTCCCGCAGCGGGCGCGATGTCACCGCCGAGTATCCGCAATTGCGGGCATTGGCGGAGGATCTCGCCGATCACCACGTGGTGCTGGACGGCGAGGCCGTCGTACTTGACTCCTCTGGTGTGCCCAGCTTCAGCCAGATGCAGAATCGGGGCCGCGACACCCGTGTCGAGTTCTGGGCGTTCGACCTGCTCTACCTCGACGGCCGCGCGCTGCTAGGCACCCGCTACCAAGACCGGCGTAAGCTGCTCGAAACCCTAGCTAACGCAACCAGTCTCACCGTTCCCGAGCTGCTGCCCGGTGACGGCGCCCAAGCGTTTGCGTGCTCGCGCAAGCACGGCTGGGAGGGCGTGATCGCCAAGAGGCGTGACTCGCGCTATCAGCCGGGCCGGCGCTGCGCGTCGTGGGTCAAGGACAAGCACTGGAACACCCAGGAAGTCGTCATTGGTGGCTGGCGCGCCGGGGAAGGCGGGCGCAGCAGTGGCGTCGGGTCGCTGCTCATGGGCATCCCCGGTCCAGGTGGGCTGCAGTTCGCCGGGCGGGTCGGTACCGGCCTCAGCGAACGCGAACTGGCCAACCTCAAGGAGATGCTGGCGCCGCTGCATACCGACGAGTCCCCCTTCGACGTACCACTGCCCGCGCGTGACGCCAAGGGCATCACATATGTCAAGCCGGCGCTGGTTGCAGAGGTGCGCTACAGCGAGTGGACTCCGGAGGGCCGGCTGCGTCAATCAAGCTGGCGTGGGCTGCGGCCGGACAAGAAACCCAGTGAGGTGGTGCGCGAATGA

SEQ ID NO: 8 - protein, 759 aa, Mycobacterium tuberculosis
MGSASEQRVTLTNADKVLYPATGTTKSDIFDYYAGVAEVMLGHIAGRPATRKRWPNGVDQPAFFEKQLALSAPPWLSRATVAHRSGTTTYPIIDSATGLAWIAQQAALEVHVPQWRFVAEPGSGELNPGPATRLVFDLDPGEGVMMAQLAEVARAVRDLLADIGLVTFPVTSGSKGLHLYTPLDEPVSSRGATVLAKRVAQRLEQAMPALVTSTMTKSLRAGKVFVDWSQNSGSKTTIAPYSLRGRTHPTVAAPRTWAELDDPALRQLSYDEVLTRIARDGDLLERLDADAPVADRLTRYRRMRDASKTPEPIPTAKPVTGDGNTFVIQEHHARRPHYDFRLECDGVLVSWAVPKNLPDNTSVNHLAIHTEDHPLEYATFEGAIPSGEYGAGKVIIWDSGTYDTEKFHDDPHTGEVIVNLHGGRISGRYALIRTNGDRWLAHRLKNQKDQKVFEFDNLAPMLATHGTVAGLKASQWAFEGKWDGYRLLVEADHGAVRLRSRSGRDVTAEYPQLRALAEDLADHHVVLDGEAVVLDSSGVPSFSQMQNRGRDTRVEFWAFDLLYLDGRALLGTRYQDRRKLLETLANATSLTVPELLPGDGAQAFACSRKHGWEGVIAKRRDSRYQPGRRCASWVKDKHWNTQEVVIGGWRAGEGGRSSGVGSLLMGIPGPGGLQFAGRVGTGLSERELANLKEMLAPLHTDESPFDVPLPARDAKGITYVKPALVAEVRYSEWTPEGRLRQSSWRGLRPDKKPSEVVRE

SEQ ID NO: 9 - protein, 868 aa, Mycobacterium tuberculosis
CDIERENDESEQENZVMPRTEINKASMYCBACTERIMTBERCLSISATGCGAGCCATTTGGACGGGTTCGATCGCCTTCGGGCTGGTGAACGTGCCGGTCAAGGTGTACAGCGCTACCGCAGACCACGACATCAGGTTCCACCAGGTGCACGCCAAGGACAACGGACGCATCCGGTACAAGCGCGTCTGCGAGGCGTGTGGCGAGGTGGTCGACTACCGCGATCTTGCCCGGGCCTACGAGTCCGGCGACGGCCAAATGGTGGCGATCACCGACGACGACATCGCCAGCTTGCCTGAAGAACGCAGCCGGGAGATCGAGGTGTTGGAGTTCGTCCCCGCCGCCGACGTGGACCCGATGATGTTCGACCGCAGCTACTTTTTGGAGCCTGATTCGAAGTCGTCGAAATCGTATGTGCTGCTGGCTAAGACACTCGCCGAGACCGACCGGATGGCGATCGTGCATTTCACGCTGCGCAACAAGACCAGGCTGGCGGCGTTGCGCGTCAAGGATTTCGGCAAGCGAGAGGTGATGATGGTGCACACGTTGCTGTGGCCCGATGAGATCCGCGACCCCGACTTCCCGGTGCTGGACCAGAAGGTGGAGATCAAACCCGCGGAACTCAAGATGGCCGGCCAGGTGGTGGACTCGATGGCCGACGACTTCAATCCGGACCGCTACCACGACACCTACCAGGAGCAGTTACAGGAGCTGATCGACACCAAACTCGAAGGTGGGCAGGCATTTACCGCCGAGGACCAACCGAGGTTGCTGGACGAGCCCGAAGACGTCTCCGACCTGCTCGCCAAGCTGGAGGCCAGCGTGAAGGCGCGCTCGAAGGCCAACTCAAACGTCCCAACGCCTCCGTGA

SEQ ID NO: 10 - protein, 273 aa, Mycobacterium tuberculosis
MRAIWTGSIAFGLVNVPVKVYSATADHDIRFHQVHAKDNGRIRYKRVCEACGEVVDYRDLARAYESGDGQMVAITDDDIASLPEERSREIEVLEFVPAADVDPMMFDRSYFLEPDSKSSKSYVLLAKTLAETDRMAIVHFTLRNKTRLAALRVKDFGKREVMMVHTLLWPDEIRDPDFPVLDQKVEIKPAELKMAGQVVDSMADDFNPDRYHDTYQEQLQELIDTKLEGGQAFTAEDQPRLLDEPEDVSDLLAKLEASVKARSKANSNVPTPP

SEQ ID NO: 11 - protein, 3296 aa, Mycobacterium tuberculosis
TCTCATGTTTGACAGCTTATCATCGAATTATAGGAATAGAGCAAACAAGCAAAGGAAATTTTGTCAAAATAATTTTATTGACAACGTCTTATTAACGTTGATATAATTTAAATTTTATTTGACAAAAATGGGCTCGTGTTGTACAATAAATGTAACTAGTTAAGGAGGTAATAATATATGGGTTCGGCGTCGGAGCAACGGGTGACGCTGACCAACGCCGACAAGGTGCTCTATCCCGCCACCGGGACCACAAAGTCCGATATCTTCGACTACTACGCCGGTGTTGCCGAAGTCATGCTCGGCCACATCGCGGGACGGCCGGCGACGCGCAAGCGCTGGCCTAACGGCGTCGACCAACCCGCGTTCTTCGAAAAGCAGTTGGCGTTGTCGGCGCCGCCTTGGCTGTCACGTGCAACGGTGGCGCACCGGTCCGGGACGACGACCTATCCGATCATCGATAGCGCAACCGGGCTGGCCTGGATCGCCCAACAGGCGGCGCTGGAGGTGCACGTGCCGCAGTGGCGGTTTGTCGCCGAGCCCGGATCAGGTGAGTTAAATCCGGGCCCGGCAACGCGTTTGGTGTTCGACCTGGACCCGGGCGAAGGCGTGATGATGGCCCAGCTGGCCGAGGTGGCGCGCGCGGTTCGTGATCTTCTCGCCGATATCGGGTTGGTCACCTTCCCGGTCACCAGCGGCAGCAAGGGATTGCATCTGTACACACCGCTGGATGAGCCGGTGAGCAGCAGGGGAGCCACGGTGTTGGCCAAGCGCGTCGCGCAGCGATTGGAGCAGGCGATGCCCGCGTTGGTCACCTCGACCATGACCAAAAGCCTGCGGGCCGGGAAGGTGTTTGTGGACTGGAGCCAGAACAGCGGCTCGAAGACCACCATCGCGCCGTACTCACTACGTGGCCGGACGCATCCGACCGTCGCGGCGCCACGCACCTGGGCGGAGCTCGACGACCCCGCACTGCGTCAGCTCTCCTACGACGAGGTGCTGACCCGGATTGCCCGCGACGGCGATCTGCTCGAGCGGCTGGATGCCGACGCTCCGGTAGCGGACCGGTTGACCCGATACCGCCGCATGCGCGACGCATCGAAAACTCCCGAGCCGATTCCCACGGCGAAACCCGTTACCGGAGACGGCAATACGTTCGTCATCCAGGAGCATCACGCGCGTCGGCCGCACTACGATTTCCGGCTGGAATGCGACGGCGTGCTGGTCTCGTGGGCGGTACCGAAAAACCTGCCCGACAACACATCGGTTAACCATCTAGCGATACACACCGAGGACCACCCGCTGGAATACGCCACGTTCGAGGGCGCGATTCCCAGCGGGGAGTACGGCGCCGGCAAGGTGATCATCTGGGACTCCGGCACTTACGACACCGAGAAGTTCCACGATGACCCGCACACGGGGGAGGTCATCGTGAATCTGCACGGCGGCCGGATCTCTGGGCGTTATGCGCTGATTCGGACCAACGGCGATCGGTGGCTGGCGCACCGCCTAAAGAATCAGAAAGACCAGAAGGTGTTCGAGTTCGACAATCTGGCCCCAATGCTTGCCACGCACGGCACGGTGGCCGGTCTAAAGGCCAGCCAGTGGGCGTTCGAAGGCAAGTGGGACGGCTACCGGTTGCTGGTTGAGGCTGACCACGGCGCCGTGCGGCTGCGGTCCCGCAGCGGGCGCGATGTCACCGCCGAGTATCCGCAATTGCGGGCATTGGCGGAGGATCTCGCCGATCACCACGTGGTGCTGGACGGCGAGGCCGTCGTACTTGACTCCTCTGGTGTGCCCAGCTTCAGCCAGATGCAGAATCGGGGCCGCGACACCCGTGTCGAGTTCTGGGCGTTCGACCTGCTCTACCTCGACGGCCGCGCGCTGCTAGGCACCCGCTACCAAGACCGGCGTAAGCTGCTCGAAACCCTAGCTAACGCAACCAGTCTCACCGTTCCCGAGCTGCTGCCCGGTGACGGCGCCCAAGCGTTTGCGTGCTCGCGCAAGCACGGCTGGGAGGGCGTGATCGCCAAGAGGCGTGACTCGCGCTATCAGCCGGGCCGGCGCTGCGCGTCGTGGGTCAAGGACAAGCACTGGAACACCCAGGAAGTCGTCATTGGTGGCTGGCGCGCCGGGGAAGGCGGGCGCAGCAGTGGCGTCGGGTCGCTGCTCATGGGCATCCCCGGTCCAGGTGGGCTGCAGTTCGCCGGGCGGGTCGGTACCGGCCTCAGCGAACGCGAACTGGCCAACCTCAAGGAGATGCTGGCGCCGCTGCATACCGACGAGTCCCCCTTCGACGTACCACTGCCCGCGCGTGACGCCAAGGGCATCACATATGTCAAGCCGGCGCTGGTTGCAGAGGTGCGCTACAGCGAGTGGACTCCGGAGGGCCGGCTGCGTCAATCAAGCTGGCGTGGGCTGCGGCCGGACAAGAAACCCAGTGAGGTGGTGCGCGAATGATAAGGAGGTAATAATATATGCGAGCCATTTGGACGGGTTCGATCGCCTTCGGGCTGGTGAACGTGCCGGTCAAGGTGTACAGCGCTACCGCAGACCACGACATCAGGTTCCACCAGGTGCACGCCAAGGACAACGGACGCATCCGGTACAAGCGCGTCTGCGAGGCGTGTGGCGAGGTGGTCGACTACCGCGATCTTGCCCGGGCCTACGAGTCCGGCGACGGCCAAATGGTGGCGATCACCGACGACGACATCGCCAGCTTGCCTGAAGAACGCAGCCGGGAGATCGAGGTGTTGGAGTTCGTCCCCGCCGCCGACGTGGACCCGATGATGTTCGACCGCAGCTACTTTTTGGAGCCTGATTCGAAGTCGTCGAAATCGTATGTGCTGCTGGCTAAGACACTCGCCGAGACCGACCGGATGGCGATCGTGCATTTCACGCTGCGCAACAAGACCAGGCTGGCGGCGTTGCGCGTCAAGGATTTCGGCAAGCGAGAGGTGATGATGGTGCACACGTTGCTGTGGCCCGATGAGATCCGCGACCCCGACTTCCCGGTGCTGGACCAGAAGGTGGAGATCAAACCCGCGGAACTCAAGATGGCCGGCCAGGTGGTGGACTCGATGGCCGACGACTTCAATCCGGACCGCTACCACGACACCTACCAGGAGCAGTTACAGGAGCTGATCGACACCAAACTCGAAGGTGGGCAGGCATTTACCGCCGAGGACCAACCGAGGTTGCTGGACGAGCCCGAAGACGTCTCCGACCTGCTCGCCAAGCTGGAGGCCAGCGTGAAGGCGCGCTCGAAGGCCAACTCAAACGTCCCAACGCCTCCGTGA

SEQ ID NO: 12 - protein, 3720 aa, Mycobacterium tuberculosis
  Vektor
TCTCATGTTTGACAGCTTATCATCGAATTATAGGAATAGAGCAAACAAGCAAAGGAAATTTTGTCAAAATAATTTTATTGACAACGTCTTATTAACGTTGATATAATTTAAATTTTATTTGACAAAAATGGGCTCGTGTTGTACAATAAATGTAACTAGTTAAGGAGGTAATAATATATGGGTTCGGCGTCGGAGCAACGGGTGACGCTGACCAACGCCGACAAGGTGCTCTATCCCGCCACCGGGACCACAAAGTCCGATATCTTCGACTACTACGCCGGTGTTGCCGAAGTCATGCTCGGCCACATCGCGGGACGGCCGGCGACGCGCAAGCGCTGGCCTAACGGCGTCGACCAACCCGCGTTCTTCGAAAAGCAGTTGGCGTTGTCGGCGCCGCCTTGGCTGTCACGTGCAACGGTGGCGCACCGGTCCGGGACGACGACCTATCCGATCATCGATAGCGCAACCGGGCTGGCCTGGATCGCCCAACAGGCGGCGCTGGAGGTGCACGTGCCGCAGTGGCGGTTTGTCGCCGAGCCCGGATCAGGTGAGTTAAATCCGGGCCCGGCAACGCGTTTGGTGTTCGACCTGGACCCGGGCGAAGGCGTGATGATGGCCCAGCTGGCCGAGGTGGCGCGCGCGGTTCGTGATCTTCTCGCCGATATCGGGTTGGTCACCTTCCCGGTCACCAGCGGCAGCAAGGGATTGCATCTGTACACACCGCTGGATGAGCCGGTGAGCAGCAGGGGAGCCACGGTGTTGGCCAAGCGCGTCGCGCAGCGATTGGAGCAGGCGATGCCCGCGTTGGTCACCTCGACCATGACCAAAAGCCTGCGGGCCGGGAAGGTGTTTGTGGACTGGAGCCAGAACAGCGGCTCGAAGACCACCATCGCGCCGTACTCACTACGTGGCCGGACGCATCCGACCGTCGCGGCGCCACGCACCTGGGCGGAGCTCGACGACCCCGCACTGCGTCAGCTCTCCTACGACGAGGTGCTGACCCGGATTGCCCGCGACGGCGATCTGCTCGAGCGGCTGGATGCCGACGCTCCGGTAGCGGACCGGTTGACCCGATACCGCCGCATGCGCGACGCATCGAAAACTCCCGAGCCGATTCCCACGGCGAAACCCGTTACCGGAGACGGCAATACGTTCGTCATCCAGGAGCATCACGCGCGTCGGCCGCACTACGATTTCCGGCTGGAATGCGACGGCGTGCTGGTCTCGTGGGCGGTACCGAAAAACCTGCCCGACAACACATCGGTTAACCATCTAGCGATACACACCGAGGACCACCCGCTGGAATACGCCACGTTCGAGGGCGCGATTCCCAGCGGGGAGTACGGCGCCGGCAAGGTGATCATCTGGGACTCCGGCACTTACGACACCGAGAAGTTCCACGATGACCCGCACACGGGGGAGGTCATCGTGAATCTGCACGGCGGCCGGATCTCTGGGCGTTATGCGCTGATTCGGACCAACGGCGATCGGTGGCTGGCGCACCGCCTAAAGAATCAGAAAGACCAGAAGGTGTTCGAGTTCGACAATCTGGCCCCAATGCTTGCCACGCACGGCACGGTGGCCGGTCTAAAGGCCAGCCAGTGGGCGTTCGAAGGCAAGTGGGACGGCTACCGGTTGCTGGTTGAGGCTGACCACGGCGCCGTGCGGCTGCGGTCCCGCAGCGGGCGCGATGTCACCGCCGAGTATCCGCAATTGCGGGCATTGGCGGAGGATCTCGCCGATCACCACGTGGTGCTGGACGGCGAGGCCGTCGTACTTGACTCCTCTGGTGTGCCCAGCTTCAGCCAGATGCAGAATCGGGGCCGCGACACCCGTGTCGAGTTCTGGGCGTTCGACCTGCTCTACCTCGACGGCCGCGCGCTGCTAGGCACCCGCTACCAAGACCGGCGTAAGCTGCTCGAAACCCTAGCTAACGCAACCAGTCTCACCGTTCCCGAGCTGCTGCCCGGTGACGGCGCCCAAGCGTTTGCGTGCTCGCGCAAGCACGGCTGGGAGGGCGTGATCGCCAAGAGGCGTGACTCGCGCTATCAGCCGGGCCGGCGCTGCGCGTCGTGGGTCAAGGACAAGCACTGGAACACCCAGGAAGTCGTCATTGGTGGCTGGCGCGCCGGGGAAGGCGGGCGCAGCAGTGGCGTCGGGTCGCTGCTCATGGGCATCCCCGGTCCAGGTGGGCTGCAGTTCGCCGGGCGGGTCGGTACCGGCCTCAGCGAACGCGAACTGGCCAACCTCAAGGAGATGCTGGCGCCGCTGCATACCGACGAGTCCCCCTTCGACGTACCACTGCCCGCGCGTGACGCCAAGGGCATCACATATGTCAAGCCGGCGCTGGTTGCAGAGGTGCGCTACAGCGAGTGGACTCCGGAGGGCCGGCTGCGTCAATCAAGCTGGCGTGGGCTGCGGCCGGACAAGAAACCCAGTGAGGTGGTGCGCGAATGATAAGTTGGCTGCTGCCACCGCTGAGCAATAACTAGCATAACCCCTTGGGGCCTCTAAACGGGTCTTGAGGGGTTTTTTGCCCATGCAACAGAAACTATAAAAAATACAGAGAATGAAAAGAAACAGATAGATTTTTTAGTTCTTTAGGCCCGTAGTCTGCAAATCCTTTTATGATTTTCTATCAAACAAAAGAGGAAAATAGACCAGTTGCAATCCAAACGAGAGTCTAATAGAATGAGGTCGAAAAGTAAATCGCGCGGGTTTGTTACTGATAAAGCAGGCAAGACCTAAAATGTGTAAAGGGCAAAGTGTATACTTTGGCGTCACCCCTTACATATTTTAGGTCTTTTTTTATTGTGCGTAACTAACTTGCCATCTTCAAACAGGAGGGCTGGAAGAAGCAGACCGCTAACACAGTACATAAAAAAGGAGACATGAACGATGCGAGCCATTTGGACGGGTTCGATCGCCTTCGGGCTGGTGAACGTGCCGGTCAAGGTGTACAGCGCTACCGCAGACCACGACATCAGGTTCCACCAGGTGCACGCCAAGGACAACGGACGCATCCGGTACAAGCGCGTCTGCGAGGCGTGTGGCGAGGTGGTCGACTACCGCGATCTTGCCCGGGCCTACGAGTCCGGCGACGGCCAAATGGTGGCGATCACCGACGACGACATCGCCAGCTTGCCTGAAGAACGCAGCCGGGAGATCGAGGTGTTGGAGTTCGTCCCCGCCGCCGACGTGGACCCGATGATGTTCGACCGCAGCTACTTTTTGGAGCCTGATTCGAAGTCGTCGAAATCGTATGTGCTGCTGGCTAAGACACTCGCCGAGACCGACCGGATGGCGATCGTGCATTTCACGCTGCGCAACAAGACCAGGCTGGCGGCGTTGCGCGTCAAGGATTTCGGCAAGCGAGAGGTGATGATGGTGCACACGTTGCTGTGGCCCGATGAGATCCGCGACCCCGACTTCCCGGTGCTGGACCAGAAGGTGGAGATCAAACCCGCGGAACTCAAGATGGCCGGCCAGGTGGTGGACTCGATGGCCGACGACTTCAATCCGGACCGCTACCACGACACCTACCAGGAGCAGTTACAGGAGCTGATCGACACCAAACTCGAAGGTGGGCAGGCATTTACCGCCGAGGACCAACCGAGGTTGCTGGACGAGCCCGAAGACGTCTCCGACCTGCTCGCCAAGCTGGAGGCCAGCGTGAAGGCGCGCTCGAAGGCCAACTCAAACGTCCCAACGCCTCCGTGA

SEQ ID NO: 13 - protein, 444 aa, Mycobacterium tuberculosis
  Vektor 3
TCAGCCTGCAGCCCATGCAACAGAAACTATAAAAAATACAGAGAATGAAAAGAAACAGATAGATTTTTTAGTTCTTTAGGCCCGTAGTCTGCAAATCCTTTTATGATTTTCTATCAAACAAAAGAGGAAAATAGACCAGTTGCAATCCAAACGAGAGTCTAATAGAATGAGGTCGACCATGATTACGGATTCACGTTTTAGAGCTAGAAATAGCAAGTTAAAATAAGGCTAGTCCGTTATCAACTTGAAAAAGTGGCACCGAGTCGGTGCTTTTTTTTTGCCTGGCGGCAGTAGCGCGGTGGTCCCACCTGACCCCATGCCGAACTCAGAAGTGAAACGCCGTAGCGCCGATGGTAGTGTGGGGTCTCCCCATGCGAGAGTAGGGAACTGCCAGGCATCAAATAAAACGAAAGGCTCAGTCGAAAGACTGGGCCTTTCGTTTTA

SEQ ID NO: 14 - protein, 445 aa, Mycobacterium tuberculosis
TCAGCCTGCAGCCCATGCAACAGAAACTATAAAAAATACAGAGAATGAAAAGAAACAGATAGATTTTTTAGTTCTTTAGGCCCGTAGTCTGCAAATCCTTTTATGATTTTCTATCAAACAAAAGAGGAAAATAGACCAGTTGCAATCCAAACGAGAGTCTAATAGAATGAGGTCGGCCAGTGAATCCGTAATCAGTTTTAGAGCTAGAAATAGCAAGTTAAAATAAGGCTAGTCCGTTATCAACTTGAAAAAGTGGCACCGAGTCGGTGCTTTTTTTTTGCCTGGCGGCAGTAGCGCGGTGGTCCCACCTGACCCCATGCCGAACTCAGAAGTGAAACGCCGTAGCGCCGATGGTAGTGTGGGGTCTCCCCATGCGAGAGTAGGGAACTGCCAGGCATCAAATAAAACGAAAGGCTCAGTCGAAAGACTGGGCCTTTCGTTTTAT

SEQ ID NO: 15 - RNA, 480 nt, Escherichia coli
  deleted bases are underlined
aagcgggaacgttacccagcgctcccgccgcgggcaagtcccacggccaccgccggtagcccacgtcactctccggccggccgtgttttaattcaggcatcccatccccgagtgccggagccctccatgcccatccacgaaatccgccatccgctgatccgccacaagctaggcctgatgcgccgtgccgacatcagcaccaagagtttccgcgaactggcccaggaggtcggcgccctgctcacttacgaggcgaccaaggacctgcccctggagaactaccggatcgagggctggtgcggcccggtggaggtggagaagatcgccggcaagaaaatcaccgtggtgcccatcctgcgcgccgggatcggcatgctcgacggcgtgctgcgactgatcccgaacgccaaggtcagcgtggtcggcctgtcgcgggacgaggaaaccctggtcgcgcacacctacgtggaaaagctggtg

(19) United States
(12) Patent Application Publication
(10) Pub. No.: US 2021/0207134 A1
(43) Pub. Date: Jul. 8, 2021

(54) RECONSTITUTION OF DNA-END REPAIR PATHWAY IN PROKARYOTES

(71) Applicant: B.R.A I.N. AG, Zwingenberg (DE)

(72) Inventors: Ümit PUL, Duisburg (DE); Jög MAMPEL, Bensheim (DE); Christian ZUREK, Lorsch (DE); Jessica REHDORF, Bensheim-Auerbach (DE); Michael KROHN, Lorsch (DE)

(21) Appl. No.: 16/065,453

(22) PCT Filed: Dec. 23, 2016
(86) PCT No.: PCT/EP2016/082551

§ 371 Date: Jun. 22, 2018

(30) Foreign Application Priority Data
Dec. 24, 2015 (EP) 15202718.1

(51) Int. Cl. C12N 15/11; C12N 9/22; C12N 15/90; C07K 14/35

(52) U.S. Cl. CPC C12N 15/11; C12N 9/22; C12N 2800/80; C07K 14/35; C12N 2310/20; C12N 15/902

(57) ABSTRACT

Suggested is a method for engineering and/or editing the genome of prokaryotes encompassing the following steps: (i) providing a culture of prokaryotic cells, (ii) preparing a vector comprising an expression system encompassing at least one programmable DNA-binding and -cleaving protein, (iii) introducing said vector into said prokaryotic cells to target a specific DNA sequence in the genome of said prokaryotic cells.

### FIELD OF INVENTION

The present invention relates to genome engineering and editing in prokaryotes, particularly targeted modification of a prokaryotic genome, such as disruption of gene function (knock-out), deletion of genomic locus or insertion of DNA elements that may use vector systems to reconstitute DNA-end repair system in prokaryotes in combination with programmable nucleases.

### STATE OF THE ART

Targeted genome engineering and editing relies on the capability to introduce precise DNA-cleavage at the genomic locus of interest and on the capability of the host cell to repair the cleavage site. Several programmable DNA-binding and -cleaving proteins have been developed that allow a precise introduction of double-strand DNA breaks (DSBs) at a specific genomic locus of interest in order to modify the DNA sequence flanking the cleavage site. Examples of such programmable DNA-cutting enzymes include Zn-finger or TAL nucleases, meganucleases and CRISPR-Cas9 [1, 2]. In eukaryotes DSBs are repaired by either endogenous non-homologous end-joining (NHEJ) or homologous recombination (HR) pathway. In the NHEJ pathway the DNA-breaks are enzymatically sealed by a set of proteins including the DNA-end binding protein Ku that recruits ligases to the cleavage site. Heterodimeric Ku protein specifically binds to the DNA-ends and mediates the repair of DSBs by promoting the formation of DNA-end synapsis and recruitment of recombination proteins, including DNA ligases. NHEJ repair is intrinsically erroneous and leads to deletion or insertion of few bases. These indel (insertion-deletion) mutations can cause frameshift mutation and thus to knockout protein encoding genes when the repair site is located within an open-reading-frame (ORF) [2]. Thus, a simple way to knock-out a gene of interest is to introduce DSB within its ORF using programmable DNA-cutting protein in order to induce the error-prone NHEJ pathway.

Due to the lack of NHEJ repair proteins in most prokaryotes, DSBs have to be repaired by homologous repair pathway, which requires the presence of a donor-template DNA that contains homologous sequences flanking the DSBs [3-5]. Otherwise, DSBs introduced in the genomic DNA (self-targeting) causes death of the prokaryotic host [3]. Therefore, the use of the DNA-cutting enzymes, like Cas9, meganucleases, TAL nucleases, Zn finger proteins for targeted gene modification in prokaryotes is coupled to the homologous recombination system and requires providing of homologous recombination template for each targeted DNA site. This limits the applicability of especially versatile CRISPR-based methods, such as multiplex genome editing and genome-wide knock-out (GeCKO) screenings [6]. As a matter of fact CRISPR-Cas9 technology is today's most promising tool for genome engineering, providing

the ability to perform multiplex gene modifications in a single cell at the same time and

the application of CRISPR-RNA libraries for genome-wide “loss-of-function” screening assays, both applicable in many eukaryotic model organisms.

Both methods cannot be transferred to prokaryotes due to the lack of NHEJ pathway. A method that reconstitutes the DNA-end repair pathway in prokaryotes would allow the adaptability of Cas9-based genome engineering/editing and genome-wide screening assays in prokaryotes.

Therefore, there is urgent need to develop vector systems and methods that reconstitutes the DNA-end repair pathway in prokaryotic host cells, which may allow the application of genome engineering and editing technologies in prokaryotic organisms.

The object of the present invention has been to overcome this limitation in prokaryotes by utilization of NHEJ and NHEJ-like repair pathways in order to reconstitute DNA-end repair system in prokaryotes

to prevent cell death caused by DSBs and

to enable the (erroneous) repair of DSBs created by programmable DNA-cleaving proteins like CRISPR-Cas9.

### DESCRIPTION OF THE INVENTION

Object of the present invention is a method for engineering and/or editing the genome of prokaryotes (bacteria or archaea) encompassing the following steps:
(i) providing a culture of prokaryotic cells,
(ii) preparing a vector comprising an expression system encompassing at least one programmable DNA-binding and cleaving protein,
(iii) introducing said vector into said prokaryotic cells to target a specific DNA sequence in the genome of said prokaryotic cells.

More particularly, the method encompasses the following steps:
(i) providing a culture of prokaryotic cells,
(ii) preparing a vector comprising an expression cassette for a DNA-double strand break repair system encompassing
(a) at least one protein binding to the DNA-ends,
(b) at least one protein with DNA-ligase activity
(iii) introducing said vector into said prokaryotic cells to enable introduction of double strand DNA breaks according to claim 1 in the genome of said prokaryotic cells.

Even more preferred is the embodiment according to which the method encompasses the following steps:
(i) providing a culture of prokaryotic cells,
(ii) designing at least one type of single-guide RNA (sgRNA), the 10 to 50 nucleotides (nt) guide sequence of said sgRNA being complementary to desired stretches within the non-coding and/or putative regulatory regions upstream of the translation start codon of at least one gene of said prokaryotic cell;
(iii) preparing a vector comprising an expression cassette encompassing
   (a) at least one programmable DNA-binding and cleaving protein,
   (b) at least one optionally modified sgRNA; and
   (c) at least one DNA-end binding protein; and
(iv) transforming said culture of prokaryotic cells with said vectors by standard methods (e.g. chemical transformation, electroporation, conjugation or transduction) to target the genome for the presence of a DNA sequence that is complementary to the 10 to 50 nt guide sequence of said sgRNA or protein-based nucleases like TAL- or Zn-finger proteins.

In this context reference is made to the paper by T. Su et al. titled “A CRISPR-Cas9 Assisted Non-Homologous End-Joining Strategy for One-step Engineering of Bacterial Genome” (www.nature.com/Scientific Reports 6:37895/D01:10.1038/srep37895), published Nov. 24, 2016, which means after the priority date the present application is assigned to. The authors describe a CRISPR-Cas9 assisted non-homologous end-joining (CA-NHEJ) strategy for the rapid and efficient inactivation of bacterial genes in a homologous recombination-independent manner and without the use of selective markers. According to this study CA-NHEJ can be used to delete large chromosomal DNA fragments in a single step without the prerequisite of a homologous DNA template. Obviously, the paper refers to the same problem and provides a similar solution, thus providing additional proof that the proposed technical teaching is effective.

### BRIEF DESCRIPTION OF THE INVENTION

For transforming a culture of prokaryotic cells with plasmid vectors standard methods which are known in the art can be applied, such as chemical transformation, electroporation, conjugation or transduction. The vector can be a plasmid, a bacteriophage, a phagemid or a virus.

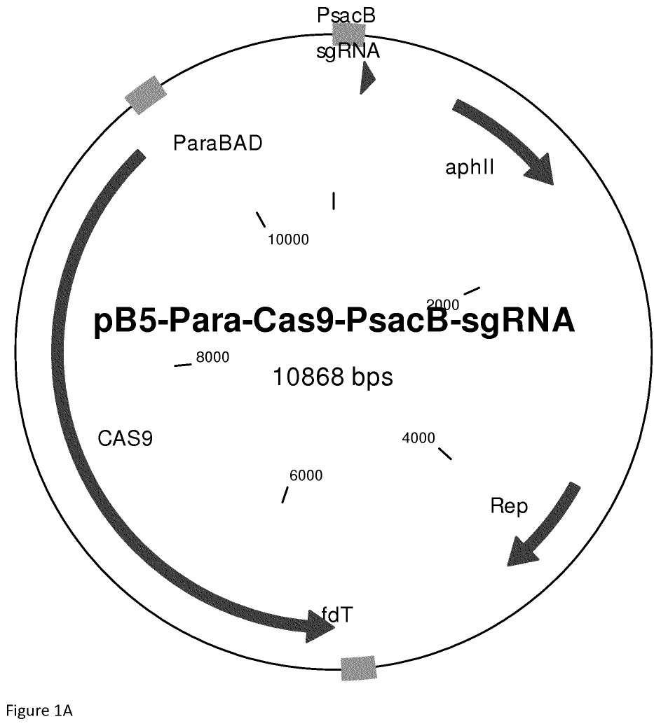

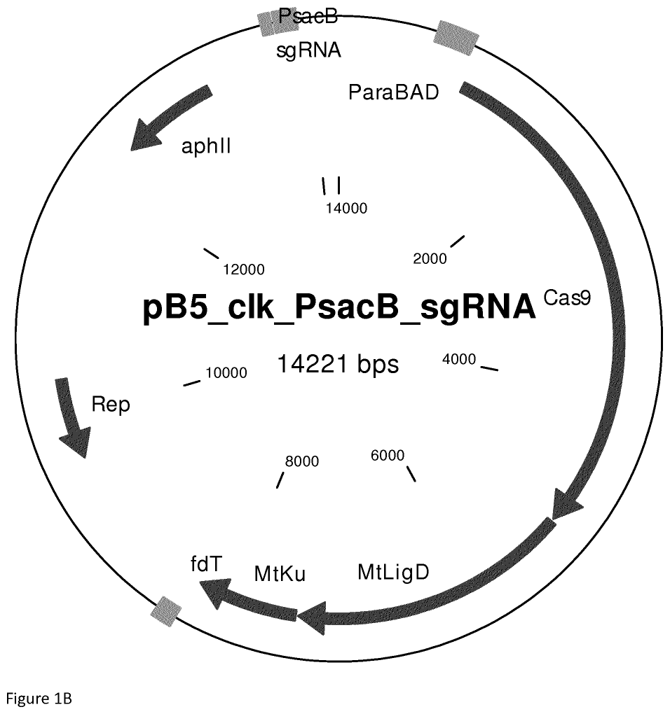

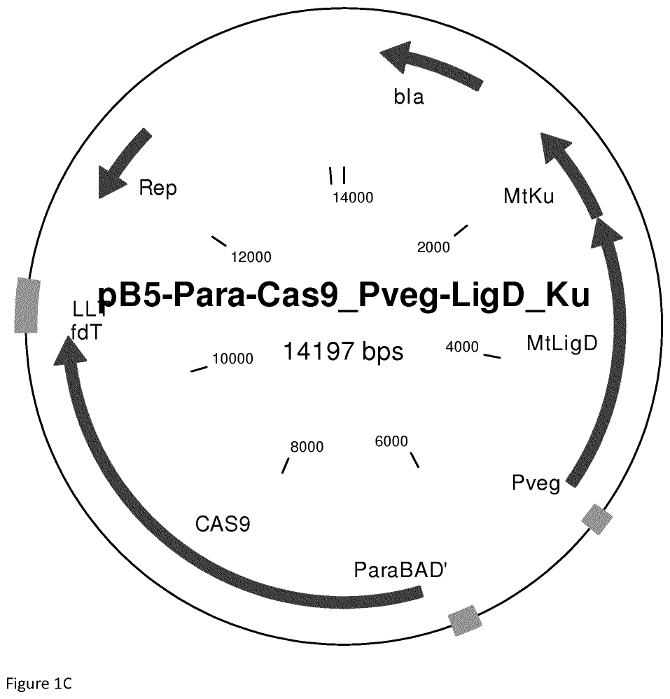

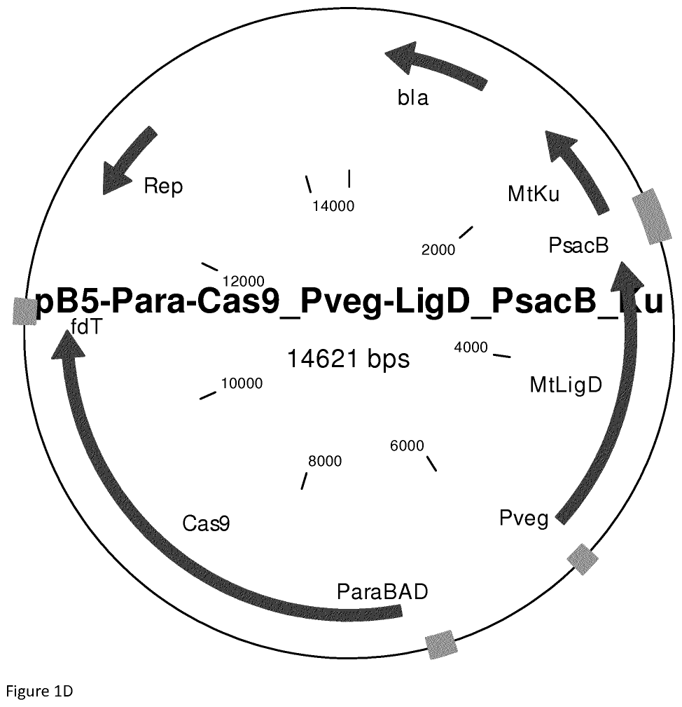

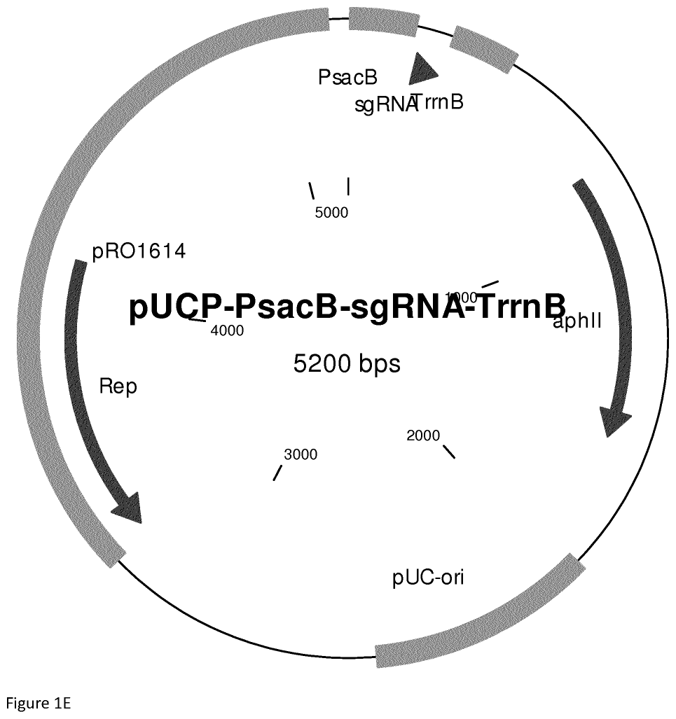

Few prokaryotic species contain genes that show functional similarity to the eukaryotic proteins involved in the NHEJ pathway. A well-known example is the NHEJ-like pathway in Mycobacterium tuberculosis mediated by the two proteins Ku (MtKu) and LigD (MtLigD). In order to proof our intention to induce the repair of DNA-ends in prokaryotes when using programmable DNA-cutting enzymes we analyzed the effect of MtKu and MtLigD on the toxicity of self-targeting Cas9-sgRNA ribonucleoproteins in Azotobacter vinelandii, Escherichia coli and Pseudomonas putida. To this aim, we designed two vectors, one that encodes the Cas9 protein (pB5-Para-Cas9-PsacB-sgRNA, FIG. 1A) and another vector that encodes Cas9, MtLigD and MtKu proteins (pB5-CLK_PsacB-sgRNA, FIG. 1B). Both vectors also comprise the expression cassette for the transcription of a sgRNA from the promoter PsacB. Using the restriction enzyme BbsI, we are able to modify the first 20 nucleotides of the sgRNA on both vectors, which determine the cleavage site by the Cas9 protein.

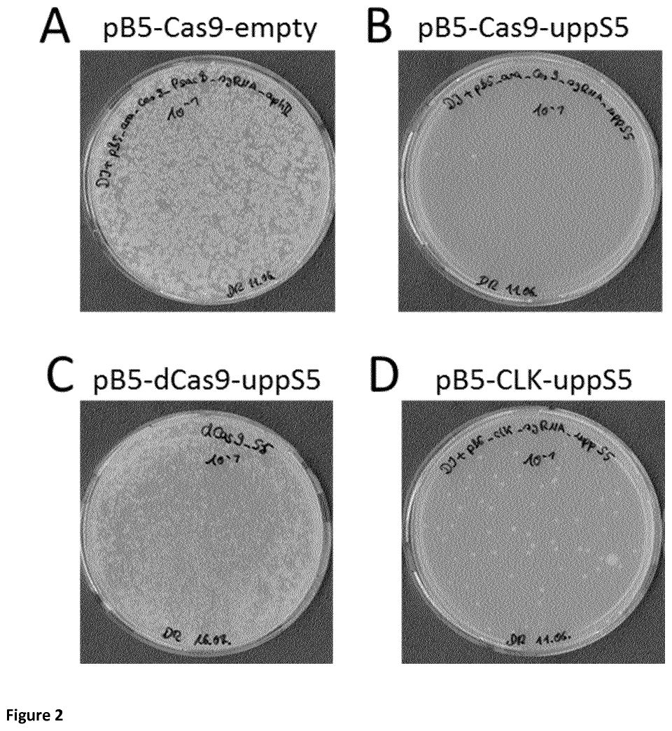

To solve the problem underlying the present invention a guide sequence into the vectors pB5-Para-Cas9-PsacB-sgRNA and pB5-CLK_PsacB-sgRNA was inserted that directs the Cas9 nuclease to the upp gene of A. vinelandii[7]. Since the upp gene is not essential, a toxicity of upp targeting Cas9 would be an indication for the detrimental effect of DSBs on cell viability per se. Indeed, the expression of upp-targeting Cas9-sgRNA complexes from the pB5-Para-Cas9-PsacB-sgRNA vector results in almost complete lack of viable A. vinelandii (compare FIGS. 2A and 2B). To proof the Cas9-specificity of the observed toxic effect two amino acids of Cas9, known to inactivate the nuclease activity of Cas9 (dCas9, D10A and H840A) were replaced. As can be seen in FIG. 2C the expression of dCas9-upp-sgRNA has no effect on the viability confirming that the toxicity is based on the DSB induced by wild-type Cas9 loaded with upp-targeting sgRNA. Next, the repair proteins MtKu and MtLigD with the self-targeting wildtype Cas9-upp-sgRNA were co-expressed. The results show clearly that in the presence of MtKu and MtLigD the number of the survived cells is significantly increased (compare FIG. 2B and FIG. 2D), indicating a promoting effect of MtKu and MtLigD on the repair of Cas9-induced DNA-cleavage in A. vinelandii. In eukaryotes up to 10% of the repair events by the NHEJ pathway results in mutation at the repair site. In order to test whether the repair with MtKu-MtLigD in A. vinelandii results in mutations at the targeted region, the viable clones on agar plates supplemented with 5-Fluorouracile (5-FU) were selected, whose presence in the growth medium is only toxic for the cells that contain an intact upp gene. Indeed, it was possible to isolate 5-FU resistant clones that were transformed with pB5-CLK_PsacB-sgRNA_upp55 vector containing a single guide sequence targeting the upp gene. Genomic DNA from 5-FU-resistent clones was prepared and a region spanning the upp gene was amplified by PCR and analyzed by Sanger sequencing.

As shown in FIG. 3, clones, which escaped the toxicity of Cas9-induced DSB at the upp gene, contain a large deletion 3-bp immediately upstream of the protospacer adjacent motif (PAM) 5″-NGG-3″. Cas9-sgRNA complexes are known to introduce DSB precisely within the target region 3″-upstream of the PAM. Therefore, the sequencing results strongly suggest that the upp gene was cleaved at the expected site by Cas9 nuclease followed by exonucleolytic degradation and sealing of the resulting DNA-ends.

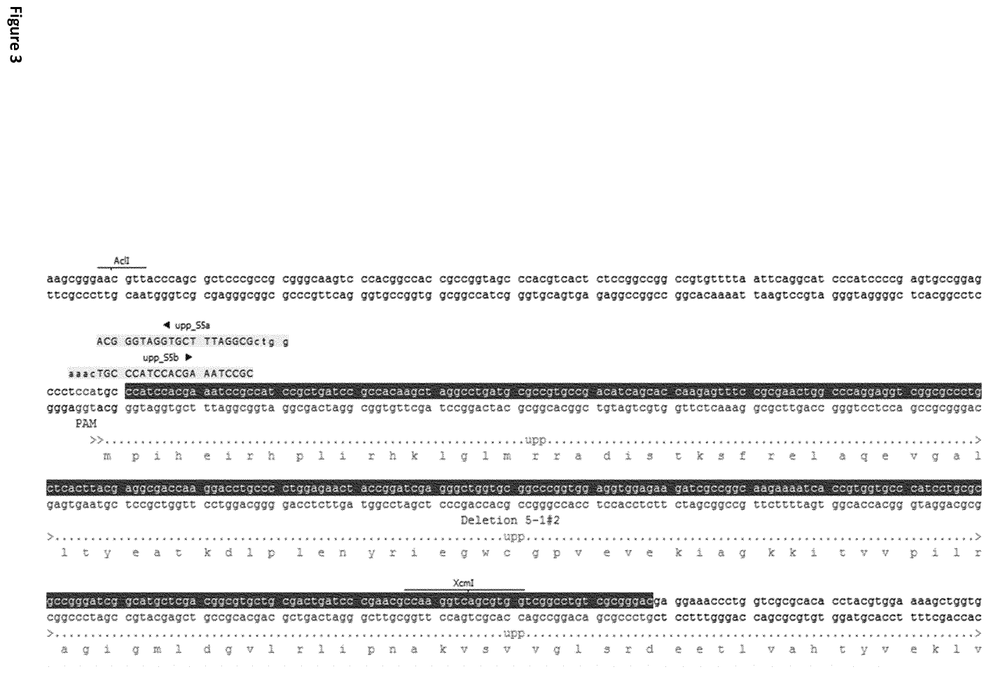

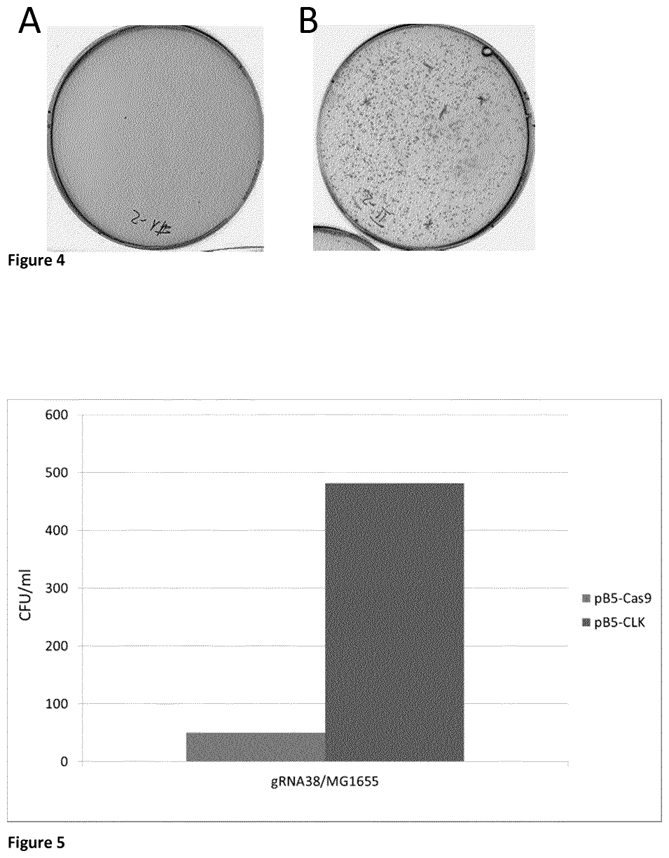

In order to show the adaptability of our system to other prokaryotic species, the experiments were repeated in Pseudomonas putida (DSM12264). Conjugation of P. putida with the vector pB5-Cas9 that targets the genomic upp gene and plating of the conjugants on selective agar plates resulted in complete lack of viable colonies (FIG. 4A). However, co-expression of MtKu and MtLigD with Cas9 and upp-targeting sgRNA resulted again in the formation of viable clones, demonstrating a reduced toxicity of Cas9-induced genomic DNA breaks in the presence of MtKu and MtLigD.

Next, similar analyses in E. coli MG1655 were performed using spacer sequences that direct Cas9 to the genomic lacZ gene. As shown in FIG. 5, the co-induction of the repair proteins MtKu and MtLigD also reduced the toxicity of self-targeting Cas9-sgRNA complexes in E. coli, indicating a wide-range applicability of our system in different prokaryotic species. Thus, based on these results, one can conclude that the Cas9 technology and other programmable nucleases that introduce DSBs can be applied in prokaryotes by coupling the nuclease activity with repair proteins that reconstitute DNA-end repair pathway.

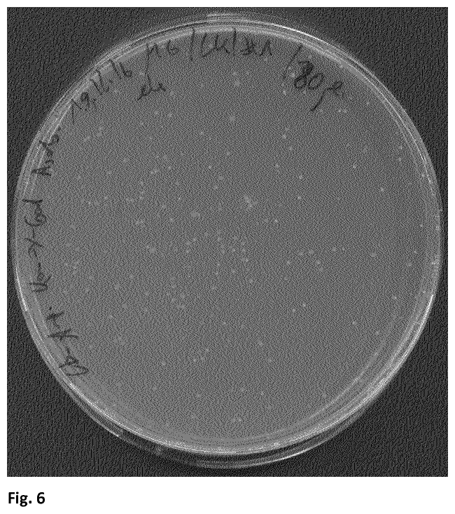

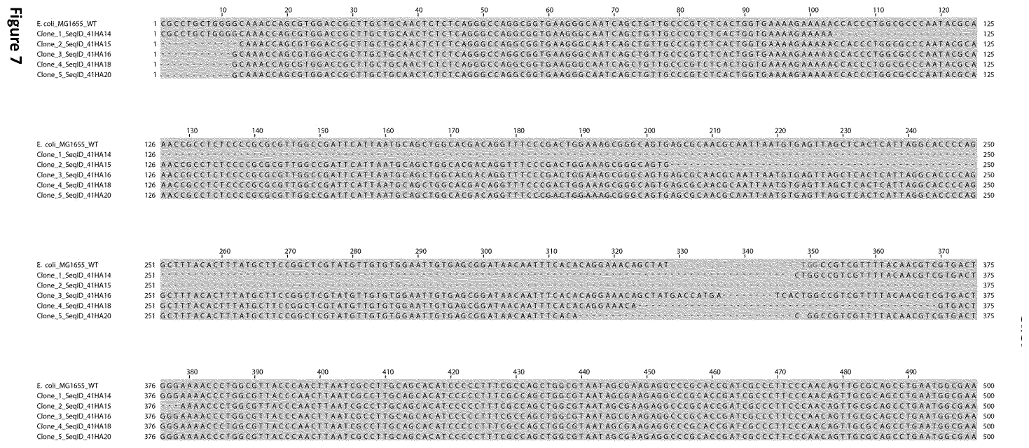

In order to test a nuclease-mediated introduction of mutations into the lacZ gene E. coli MG1655 was transformed either with the plasmid pB5-Para-Cas9-Pveg-LigD_Ku (FIG. 1C) that encodes for ParaBAD-driven Cas9, Pveg-driven LigD-Ku or with pB5-Para-Cas9-PvegLigD_Psac_Ku that encodes for ParaBAD-driven Cas9, Pveg-driven LigD and PsacB-driven Ku proteins. The cleavage of the lacZ gene was induced through a second transformation step by electroporation of the plasmid pUCP-PsacB-sgRNA-bgaI (FIG. 1E) containing the lacZ-targeting sgRNA transcription unit. The transformants were plated onto agar plates supplemented with ampicillin (100 μg/ml), kanamycin (25 μg/ml), arabinose (0.2% w/v) and X-Gal (80 μg/ml) (one example is shown in FIG. 6). Up to 24 white colonies were picked and used for single-colony PCR amplification and Sanger sequencing of the lacZ region (forward primer: 5″-GATACGACGATACCGAAGACA-3′; reverse primer: 5″-GATAACTGCCGTCACTCCAG-3′). Sequencing results of five of such clones are shown in FIG. 7, demonstrating the deletion of eight (Clone3_SeqID_41 HA16) up to 243 base-pairs (Clone 1_SeqID_41 HA14) around the Cas9-cleavage site (sgRNA target region is indicated in blue, protospacer-adjacent motif in red). The present invention enables to introduce mutations into E. coli genome without the need of homologous recombination template, which is otherwise essential to modify the E. coli genome with CRISPR-Cas9 technology [3].

### Further Objects of the Invention

Typically, the prokaryotic cells belong to bacteria or archaea, preferably bacteria.

The preferred vector is a plasmid or phage-DNA, which is usually introduced into the prokaryotic cell by means of transformation, transduction or conjugation

The programmable DNA-binding and cleaving proteins are preferably selected from the group consisting of Zn-finger, TAL nucleases, meganucleases and RNA-dependent CRISPR-associated nucleases, and more preferably from the group of CRISPR-Cas proteins belonging to class 2-type II CRISPR systems.

The most preferred programmable DNA-binding and cleaving proteins are Cas9 or Cpf1.

On the other side, the preferred DNA-end repair proteins are selected from the group consisting of proteins showing at least 30% identity in their primary sequence to protein Ku, and/or LigD of prokaryotes. The most preferred embodiment refers to DNA-end repair proteins which are selected from the group consisting of proteins Ku and/or LigD encoded by Gram-positive bacteria, more preferred encoded by Mycobacteria and particularly encoded by Mycobacterium tuberculosis.

Another object of the present invention refers to an expression system comprising
(a) at least one Cas9, modified Cas9 or Cpf1 protein,
(b) at least one optionally modified sgRNA or crRNA; and
(c) protein Ku and/or LigD.

Finally, other objects of the present invention cover:
(I) A vector comprising or consisting of the expression system as explained above.
(II) A process for gene knock-out, gene or single base pair deletion, replacement, editing, and/or for genome wide knock-out screening in a prokaryote encompassing the methods explained above.
(III) The use of DNA-end binding and -repair proteins in a process for genome engineering and editing in prokaryotes, particularly targeted modification of a prokaryotic genome, such as disruption of gene function (knock-out), deletion of genomic loci or insertion of DNA elements in prokaryotes in combination with programmable nucleases that work via introduction of DNA-double strand breaks.

### CITED REFERENCES

[1] HU J H et al. “Chemical Biology Approaches to Genome Editing: Understanding, Controlling and Delivering Programmable Nucleases”, Cell Chem. Biol. 23: 47-73 (2016)
[2] HSU P D et al. “Development of CRISPR-Cas9 for genome engineering”, Cell 157:1262-78 (2014)
[3] JIANG W et al. “RNA-guided editing of bacterial genomes using CRISPR-Cas9 systems” Nat. Biotechnol 31:233-9 (2014)
[4] LI Y et al. “Metabolic engineering of E. coli genome via the CRISPR-Cas9 mediated genome editing” Metab. Engin. 31:13-21 (2015)
[5] SHALEM 0 ET AL. “Genome-scale CRISPR Cas-9 knockout screening in human cells” SCIENCE 343: 84-7 (2014)
[6] SETUBAL J C et al. “Genome sequence of Azotobacter vinelandii, an obligate aerobe spcialized to support diverse anaerobic metabolic processes” J. Bacteriol. 191: 4534-45 (2009)

### EXAMPLES

### Example 1

As shown in FIG. 2 the presence of Ku-LigD promotes the repair of DSB induced by Cas9 loaded with self-targeting sgRNA in A. vinelandii. Isolation of clones and sequencing of the targeted region showed a specific Cas9-induced DNA a break 3-nt upstream of the PAM sequence, exonucleolytic degradation and ligation of the DNA-ends as depicted in FIG. 3.

More particularly FIG. 2 shows:
(A) A. vinelandii transformed with the plasmid pB5-Para-Cas9-PsacB-sgRNA-empty coding for Cas9 and a sgRNA without specific guide sequence. The transformants were plated on an agar plate containing Kanamycin.
(B) As in (A) but transformed with the plasmid pB5-Para-Cas9-PsacB-sgRNA-uppS5 that codes for Cas9 and sgRNA targeting the upp gene.
(C) As in (B) but transformed with the plasmid pB5-Para-dCas9-PsacB-sgRNA-uppS5 that codes for catalytically inactive Cas9 and sgRNA targeting the upp gene.
(D) As in (B) but transformed with the plasmid pB5-CLK_PsacB-sgRNA-uppS5 that encodes for Cas9-MtLigD-MtKu and sgRNA targeting upp gene.

The delivery of said plasmids into A. vinelandii was achieved by conjugation using E. coli S17-1λpir as donor cells.

A. vinelandii treated with pB5-CLK_PsacB-sgRNA-uppS5 were incubated on agar plates supplemented with 5-FU in order to select for upp mutants. Genomic DNA of a 5-FU resistant clone was isolated and the upp region was amplified by PCR. Results of Sanger sequencing showed the deletion of 308 bp (indicated in red in the sequence) region of the upp gene (FIG. 3).

### Example 2

As shown in FIG. 4 the presence of Ku-LigD promotes the repair of DSB induced by Cas9 loaded with self-targeting sgRNA in P. putida.

More particularly FIG. 4 shows:
(A) P. putida transformed with the plasmid pB5-Para-Cas9-PsacB-sgRNA-uppS15 encoding Cas9 and sgRNA targeting the upp gene.
(B) As in (A) but transformed with the plasmid pB5-CLK_PsacB-sgRNA-uppS5 that encodes Cas9-MtLigD-MtKu and sgRNA targeting upp gene.

The delivery of said plasmids into P. putida was achieved by conjugation using E. coli S17-1λpir as donor cells.

### Example 3

The presence of Ku and LigD from M. tuberculosis reduces the toxicity of self-targeting Cas9 nuclease in E. coli MG1655 (FIG. 5) and enables efficient introduction of NHEJmutations as shown in FIGS. 6 and 7.

More particularly FIG. 5 shows:

Chemically competent E. coli MG1655 was transformed either with pB5-Para-Cas9-PsacBsgRNA-bgaI or pB5-CLK_PsacB-sgRNA-bgaI. Both vectors encode wildtype Cas9 and a sgRNA targeting the lacZ gene. The vector pB5-CLK_PsacB-sgRNA-bgaI also expresses the proteins LigD and Ku from M. tuberculosis. The transformants were plated on selective agar plates and the numbers of colony forming units were determined.

FIG. 6 shows:

Chemically competent E. coli MG1655 were transformed either with pB5-Para-Cas9_PvegLigD_Ku or pB5-Para-Cas9_Pveg-LigD_PsacB_Ku. Both vectors encode wildtype Cas9, a sgRNA targeting the lacZ gene and express the proteins LigD and Ku from M. tuberculosis. Single colonies of the transformants were cultivated for preparation of electrocompetent cells.

After electroporation of the plasmid pUCP-PsacB-sgRNA-bgaI, containing lacZ-targeting sgRNA transcription unit, the transformants were plated on selective agar plates supplemented with ampicillin (100 μg/ml), kanamycin (25 μg/ml), 0.2% (w/v) arabinose and X-gal (80 μg/ml). Frameshift mutations of lacZ-gene lead to white colored colonies.

FIG. 7 shows sequencing results of wildtype lacZ gene and five NHEJ-mutants obtained with Cas9 cleavage and subsequent repair by MtKu and MtLigD. The target site of Cas9 is shown in blue, the protospacer adjacent motif in red.

FIG. 1A shows the vector maps of pB5-Para-Cas9-PsacB-sgRNA, coding for the Cas9 protein and Psac-driven sgRNA, as used for the experiments with E. coli, P. putida and A. vinelandii.

FIG. 1B shows the vector maps of pB5-CLK_PsacB_sgRNA, coding for proteins Cas9, LigD and Ku, and Psac-driven sgRNA as used for the experiments with E. coli, P. putida, A. vinelandii.

FIG. 1C shows the vector maps of pB5-Para-Cas9_Pveg-LigD_Ku, as used for knockout of lacZ-gene in E. coli.

FIG. 1D shows the vector maps of pB5-Para-Cas9_Pveg-LigD_PsacB_Ku, as used for knock-out of lacZ-gene in E. coli.

FIG. 1E shows the vector maps of pUCP-PsacB-sgRNA-TrrnB, as used for knock-out of lacZ-gene in E. coli.

1. A method for engineering and/or editing the genome of prokaryotes comprising the following steps:
(i) providing a culture of prokaryotic cells,
(ii) preparing a vector comprising an expression system encompassing at least one programmable DNA-binding and cleaving protein, and
(iii) introducing said vector into said prokaryotic cells to target a specific DNA sequence in the genome of said prokaryotic cells.

2. A method for reconstituted DNA-end repair in a prokaryote comprising the following steps:
(i) providing a culture of prokaryotic cells,
(ii) preparing a vector comprising an expression cassette for a DNA-double strand break repair system comprising
(a) at least one protein binding to the DNA-ends, and
(b) at least one protein with DNA-ligase activity, and
(iii) introducing said vector into said prokaryotic cells to enable introduction of double strand DNA breaks according to claim 1 in the genome of said prokaryotic cells.

3. A method for reconstituted DNA-end repair in a prokaryote comprising the following steps:
(i) providing a culture of prokaryotic cells,
(ii) designing at least one type of single-guide RNA (sgRNA), the 10 to 50 nucleotides (nt) guide sequence of said sgRNA being complementary to desired stretches within the non-coding and/or putative regulatory regions upstream of the translation start codon of at least one gene of said prokaryotic cell,
(iii) preparing a vector comprising an expression cassette comprising
(a) at least one programmable DNA-binding and cleaving protein,
(b) at least one optionally modified sgRNA, and
(c) at least one DNA-end binding protein, and
(iv) transforming said culture of prokaryotic cells with said vectors by standard methods (e.g. chemical transformation, electroporation, conjugation or transduction) to target the genome for the presence of a DNA sequence that is complementary to the 10 to 50 nt guide sequence of said sgRNA or protein-based nucleases like TAL- or Zn-finger proteins.

4. The method of claim 1, wherein the prokaryotic cells belong to bacteria.

5. The method of claim 1, wherein the vector is a plasmid or phage-DNA.

6. The method of claim 1, in which the vector is introduced into the prokaryotic cell by means of transformation, transduction or conjugation.

7. The method of claim 1, wherein the programmable DNA-binding and cleaving protein is selected from the group consisting of Zn-finger, TAL nucleases, meganucleases and RNA-dependent CRISPR-associated nucleases and mixtures thereof.

8. The method of claim 7, wherein the programmable DNA-binding and cleaving protein is selected from the group of CRISPR-Cas proteins belonging to class 2-type II CRISPR systems.

9. The method of claim 7, wherein the programmable DNA-binding and cleaving protein is Cas9 or Cpf1.

10. The method of claim 1, wherein the DNA-end repair protein is selected from proteins showing at least 30% identity in their primary sequence to protein Ku, and/or LigD of prokaryotes.

11. The method of claim 1, wherein the DNA-end repair protein is selected from proteins Ku and/or LigD encoded by Gram-positive bacteria.

12. The method of claim 1, wherein the DNA-end repair protein is proteins are selected from proteins Ku and/or LigD encoded by Mycobacteria.

13. The method of claim 1, wherein the DNA-end repair protein is selected from proteins Ku and/or LigD encoded by Mycobacterium tuberculosis.

14. An expression system, comprising
(a) at least one Cas9, modified Cas9 or Cpf1 protein,
(b) at least one optionally modified sgRNA or crRNA, and
(c) protein Ku and/or LigD.

15. A vector comprising the expression claim 14.

16. A process for gene knock-out, deletion, replacement, editing, and/or genome wide knock-out screening in a prokaryote, comprising utilizing the method of claim 1.

17. A process for genome engineering and editing in prokaryotes, particularly targeted modification of a prokaryotic genome, such as disruption of gene function (knock-out), deletion of genomic loci or insertion of DNA elements in prokaryotes in combination with programmable nucleases that work via introduction of DNA-double strand breaks, comprising utilizing DNA-end binding and -repair proteins.
